(19) United States
(12) Patent Application Publication
(10) Pub. No.: US 2023/0263427 A1
(43) Pub. Date: Aug. 24, 2023

(54) AN APPARATUS FOR COLLECTING BREATH

(71) Applicant: NATIONAL UNIVERSITY OF SINGAPORE, Singapore (SG)

(72) Inventors: Gokula Krishnan RAMACHANDRAN, Singapore (SG); Chen Hua YEOW, Singapore (SG)

(21) Appl. No.: 18/024,162

(22) PCT Filed: Sep. 6, 2021
(86) PCT No.: PCT/SG2021/050537

§ 371 Date: Mar. 1, 2023

(30) Foreign Application Priority Data
Sep. 8, 2020 (SG) 10202008714X

(51) Int. Cl. A61B 5/097

(52) U.S. Cl. CPC A61B 5/097

(57) ABSTRACT

Disclosed is an apparatus for collecting part of an exhaled breath. The apparatus comprises a chamber comprising an internal surface defining a sample collection volume that has been functionalised to not interact chemically or physically with volatile components in the breath, an inlet end and outlet end through which the breath respectively enters and exits the sample collection volume. The apparatus also includes two closures, one at each of the inlet end and the outlet end to control passage of the breath through the respective inlet end or outlet end, the closures being actuated to capture the part of the exhaled breath, and a sampling port accessible from outside the chamber and in communication with the sample collection volume. The chamber is further tapered in its diameter toward each of the inlet end and outlet end to maintain laminar flow through the chamber.

### TECHNICAL FIELD

The present invention relates to an apparatus for collecting breath. In particular, the present invention may relate to an apparatus for collecting end tidal breath.

### BACKGROUND

During a normal human exhalation processes, part of the exhaled breath undergoes gaseous exchange in the lungs and part does not. Parts that have not undergone gaseous exchange may not contain crucial chemical information for disease diagnosis. Moreover, those parts dilute the chemical information of parts of the exhaled breath that have undergone gaseous exchange in the lungs.

In addition, breath analysis is performed to identify and/or characterise volatile components in the collected breath. However, current breath collection apparatuses—e.g. a commercially available breath bag—are often unable to retain volatile components for long without reacting with those components. This is particularly problematic if there is a delay between collection and breath analysis, such as when a sample is collected in one location and must be analysed at another, distant location that may take some days to reach.

It is desirable therefore to provide an apparatus for collecting breath that reduces or avoids the diluting effect of parts of an exhaled breath that have not undergone gaseous exchange in the lungs, and/or enables longer storage of breath before analysis, and/or at least provides a useful alternative.

### SUMMARY OF THE INVENTION

The present disclosure provides an apparatus for collecting part of an exhaled breath, comprising:
a chamber comprising:
   an internal surface defining a sample collection volume, that has been functionalised so that it does not interact chemically or physically with volatile components in the breath;
   an inlet end through which the breath enters the sample collection volume; and
   an outlet end through which the breath exits the sample collection volume;
   two closures, one at each of the inlet end and the outlet end to control passage of the breath through the respective inlet end or outlet end, the closures being actuated to capture the part of the exhaled breath; and
   a sampling port accessible from outside the chamber and in communication with the sample collection volume,
      wherein the chamber has a longitudinal extent, and has a taper in its diameter toward each of the inlet end and outlet end.

The remainder of the Summary of the Invention section and the detailed description have been described with reference to end tidal breath. However, it will be appreciated that similar teachings apply where the apparatus is arranged to receive gases other than breath, or parts of the breath other than the end tidal part.

The internal surface may have been functionalised by being silanized.

The chamber may be formed from glass. The chamber may alternatively be formed from chemically inert materials

The sample collection volume may be a predetermined internal volume that is less than a full exhalation volume (i.e. full volume of an exhaled breath). The predetermined volume may be equal to an expected volume of the part of the exhaled breath being captured. The predetermined volume may be selected based on an age, size, smoker status or respiratory condition of a subject from whom the part of the exhaled breath is being captured. The predetermined volume may be between about 30 mL and about 400 mL. The predetermined volume may be between about 70 mL and about 350 mL. The predetermined volume may be about 70 mL. The predetermined volume may be between about 30% to about 90% of a full exhalation volume of the human or animal from which the breath (being used interchangeably herein with “part of the exhaled breath” as dictated by context, for ease of understanding, and being breath that has undergone gaseous exchange in the lungs) is being collected. The predetermined volume may be between about 50% to about 80% of a full exhalation volume (i.e. total volume of an exhaled breath) of the human or animal from which the breath is being collected. The predetermined volume may be about 70% of a full exhalation volume of the human or animal from which the breath is being collected.

The closure at the inlet end may comprise a valve. The valve may be a one-way valve to permit passage of breath into the chamber and to prevent passage of breath out of the chamber through the inlet end. The closure at the outlet end may comprise a valve.

At least one of the two closures may comprise a vacuum stopper. Each of the two closures may comprise a vacuum stopper.

The closure at the outlet end may operate independently of the closure at the inlet end.

The sampling port may be formed in one of the two closures. The sampling port may be disposed between the inlet end and the outlet end. The sampling port may comprise a gas-tight septum.

The chamber may be substantially cylindrical, having a longitudinal axis extending through both the inlet end and outlet end. The inlet end and outlet end may be at opposite ends of the chamber relative to the longitudinal axis. In being substantially cylindrical, the chamber has a generally circular cross-section—the term “generally” or “substantially” referring to there being a cylindrical body, or body with a circular cross-section, that has some discontinuities to that cylindrical or circular characteristics such as where a sampling port is located between the inlet end and outlet end and disturbs the continuity of the cylindrical or circular characteristic as described with reference to FIG. 1.

Advantageously, by functionalising the internal surface of the chamber so that it does not interact chemically or physically with volatile components in end tidal breath, those volatile components can remain unreacted for longer. This increases the period of time the breath can be stored in the sample collection volume, before it degrades—e.g. by reaction of volatile components with the apparatus, by proteins and other molecules sticking to internal surfaces and components of the apparatus, or through oxidation—sufficiently that subsequent analysis is inaccurate.

Apparatuses disclosed herein leverage the realisation that a significant proportion of an exhaled breath consists of breath stored between nostril and tracheal opening to lung (i.e., in the windpipe) does not undergo gaseous exchange in the lungs which is called dead space air. When a person exhale via mouth, the initial part of the exhaled breath is the dead space air. By opening the closures at both ends of the chamber, an exhaled breath can pass through the chamber until the dead space air has been evacuated from the chamber. At this time the outlet closure can be closed and the inlet closure is then closed to store substantially only that portion of the exhaled breath, or part thereof, that has undergone gaseous exchange in the lungs.

### BRIEF DESCRIPTION OF THE DRAWINGS

Some embodiments will not be described by way of non-limiting example only, with reference to the accompanying drawings in which:

FIG. 1 illustrates a breath collection device in accordance with present teachings;

FIG. 2 is a partial view of the breath collection device of FIG. 1, illustrating the taper at each end of the breath collection device;

FIG. 3 illustrates silanisation of the internal glass surface of a sample collection chamber in accordance with present teachings; and

FIG. 4 outlines an experimental procedure used in applying the apparatus of FIG. 1 in the detection of biomarkers for ill-health (disease) conditions in human breath.

### DETAILED DESCRIPTION

As used herein, and unless context dictates otherwise, the term “end tidal” and similar refer to breath that has undergone gaseous exchange in the lungs.

A normal human exhales approximately 500 mL of air during each exhalation. In that 500 mL volume, it may be that only the last 350 mL of air (i.e. 70% of the exhaled breath) is the resultant volume from the gaseous exchange in the lungs. It is this resultant volume that contains the crucial chemical information for disease diagnosis. Known breath bags collect the entire breath which leads to the dilution of end-tidal gas. The breath that has not undergone gaseous exchange in the lungs thus increases background noise. In many instances, the result of analysis is mostly reflects background noise.

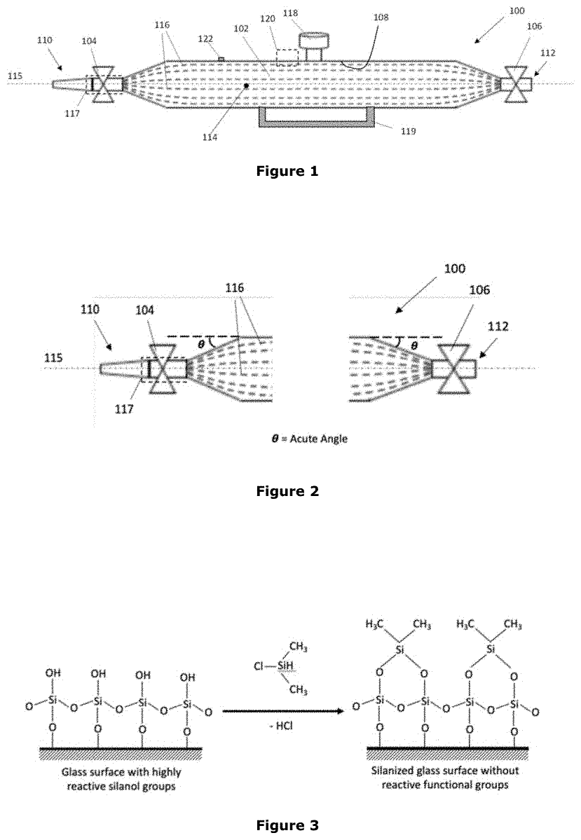

An apparatus 100 is shown in FIG. 1, for collecting part of an exhaled breath. For present purposes, the apparatus 100 will be discussed with reference to the collection of end tidal breath, though it will be understood that it may similarly be arranged to receive other gases or other parts of the exhaled breath. The apparatus 100 may thereby collect only that portion (i.e. part) of the breath that has undergone gaseous exchange within the lungs of a subject. The apparatus 100 broadly comprises a chamber 102 and two closures 104, 106, though more closures may be provided as desired, or closures 104, 106 may be combined into a single mechanism providing both inlet and outlet.

The chamber 102 has an internal surface 108, an inlet end 110 and an outlet end 112. The internal surface 108 defines a sample collection volume 114. The volume of the sample collection volume 114 may be selected or customised for trapping the end tidal portion of the breath of the subject. For example, for an adult human subject the sample collection volume may be 350 mL. For an infant the sample collection volume may be 100 mL. For an animal the volume may be different and so on. In any case, the sample collection volume is a predetermined internal volume that is less than a full exhalation volume, where the apparatus 100 is used for capturing breath. The predetermined volume may be between about 30 mL and about 400 mL. The 30 mL capacity or volume would capture the very end of an adult human end tidal breath, or any other 30 mL portion of that breath. The 400 mL would capture the full end tidal portion of the breath and a small volume from air that has not undergone gaseous exchange in the lungs. The predetermined volume may instead be between about 70 mL and about 350 mL. The predetermined volume may desirably be about 70 mL.

The internal surface 108 has been functionalised so that it does not react chemically or physically with volatile components in end tidal breath. For example, the chamber may be formed from glass—e.g. be a glass bulb—and the internal surface may be functionalised by being silanized (siliconized). The chamber may also be formed from other materials that are capable of being silanised such as metal oxides and other materials. Moreover, in stating the chamber is formed from, for example, glass, the chamber may also comprise other materials. In one embodiment, the chamber is formed from glass by having a glass bulb that has been coated by another material or sits within a protective outer shell. The outer shell may be made from a shock absorbing material such as silicon or rubber.

In normal use, air or breath enters the sample collection volume 114 through the inlet 110. Similarly, air or breath exits the sample collection volume 114 through the outlet 112. The apparatus 100 may be used to sample various portions of the exhaled breath—e.g. the very end of the end tidal breath, or the start of the end tidal breath, or the start of the whole breath such as to capture that portion of the breath that has not undergone gaseous exchange in the lungs of the subject (e.g. patient from whom the end tidal breath is being taken)—by coordinating opening and closing of closures 104, 106.

The two closures 104, 106 are positioned one at each of the inlet end 108 and the outlet end 110 to control passage of breath through the respective inlet end 108 or outlet end 110. The closure 104 at the inlet end 108 presently comprises a valve. The valve is a one-way valve to permit passage of breath into the chamber 102 and to prevent passage of breath out of the chamber 102 through the inlet end 108. The closure 106 at the outlet end 112 may similarly include a valve. In addition, the inlet end 110 itself may include a one-way valve (shown in broken lines 117) so that air can only flow into the apparatus 100 through the inlet end 110, and cannot exit the inlet end 110. The outlet end 112 may include a one-way valve so that air can only flow out of the apparatus 100 through the outlet end 112, and cannot enter the outlet end 112.

At least one, as presently both, of the two closures 102, 104 comprises a vacuum stopper. The closure 106 at the outlet end 112 can operate independently of the closure 104 at the inlet end 110. This enables the closures 104, 106 to be independently actuated to control capture of different portions of the breath, to select the precise time at which collection should begin—e.g. by allowing breath to flow through the chamber 102 until collection is desired to start—and so on.

The chamber 102 has a longitudinal extent and has a taper in its diameter toward each of the inlet end and outlet end. The term “longitudinal extent” refers to the chamber 102 having a visibly longer dimension in one axis than in other axes—presently, the longitudinal extent is along longitudinal axis 115. For example, the longitudinal extent may be used to define a longitudinal axis extending through both the inlet end and outlet end.

The chamber 102 also tapers towards both ends 110, 112, which requires the chamber 102 to have a smaller diameter at each end 110, 112 than somewhere—e.g. the chamber 102 is larger in the middle so that it can taper (reduce in diameter) towards each of the inlet end 110 and outlet end 112. If the inlet end 110 an outlet end 112 are positioned sufficiently near each other that a single mechanism can provide the function of both closures 104, 106, then the chamber 102 may simply taper towards that mechanism and thereby taper towards both the inlet end 110 and outlet end 112.

The slope of the taper—the angle of the taper relative to the longitudinal extent of the chamber 102—is selected to maintain laminar flow of breath through the chamber as shown in broken lines (some of which are referenced “116”) in FIG. 1.

In the embodiment illustrated in FIG. 1, the apparatus 100 further comprises a sampling port 118. The sampling port 118 is used to take a sample of the contents of the sample collection volume 114. The sampling port 118 is accessible from outside the chamber 102 and in communication with the sample collection volume 114. The sampling port 118 is presently shown disposed between the inlet end 110 and outlet end 112, but may instead be formed in one of the two closures 104, 106. For example, the sampling port may be incorporated into a closure by forming the closure from material that can be perforated by a needle, for extracting a sample, and that will resiliently close the perforation on retraction of the needle from the closure.

To avoid escape of the contents of the chamber 102 when not in use, the sampling port 118 comprises a gas-tight septum.

Thus, in the embodiment shown in FIG. 1, the apparatus 100 has five main components, namely a sampling port 118, high vacuum inlet stopper incorporated into the closure 104 along with the one-way valve, a chamber 102 comprising or being a glass bulb, a sampling port 118 and high vacuum outlet stopper incorporated into the outlet closure 106. The sampling port 118 itself comprises an airtight screw cap with septa through which the sample can be accessed, and may be disposed at any location along the apparatus 100 though will generally be centrally located. The inlet may also comprise a one-way valve to stop backflow of breath sample during sample collection. As mentioned above, the design of the glass bulb chamber 102 involves a number of factors, such as the slope or taper, between the inlet tube or inlet end 110 and the sample collection volume 114 of the glass bulb chamber 102, and also between the outlet tube or outlet end 106 and the sample collection volume 114 of the glass bulb chamber 102. This taper should have an acute angle (ideally between 30°-60°) as indicated by angle θ in FIG. 2 in order to reduce the air recirculation phenomenon which is caused due to turbulence created by the collision of sample air against the glass surface at the inlet and outlet.

Another important factor is the silanization of the glass bulb or chamber 102. Silanization deactivates the highly reactive silanol groups in the glass surface (FIG. 3). If silanization is not performed, these silanol groups will react with the polar gas compounds and affect the outcome of experiments or analyses conducted on the collected sample.

Silanization is performed using Dimethyl-dichloro silane (DMCS) reagent, though another organofunctional alkoxysilane molecule may be used. If silanization is not performed, these silanol groups will react with the polar gas compounds and thus affect the outcome of the experiment. Without silanization the glass surface will either bind to important volatiles of interest and reduce the effectiveness of the outcome or could chemically react with the compounds of interest and chemically modify them which could result in the wrong interpretation. This problem of extreme reactivity of the untreated glass surface defeats the purpose of using glass breath collection devices. Silanization treatment could effectively block all the highly reactive chemical groups on the internal surface of the chamber and aid in the complete and unaltered recovery of the collected volatile compounds.

In use, when the subject blows air through the one-way valve of closure 104, the air travels through the glass bulb of chamber 102 and exits through the outlet stopper of closure 106. Once exhalation is near completion, the outlet stopper of closure 106 will be closed first, followed by the closure of the inlet stopper at inlet closure 104, which will lead to the trapping the end-tidal breath. The trapped breath can be sampled from the sampling port 114, which is lined with gas tight replaceable Teflon septa. This sampling port 118 is simple and compatible with most of the volatile compound analyzing instruments like solid-phase micro-extraction fibers (SPME), gas chromatography and mass spectrometry (GC-MS), selected ion flow-tube and mass spectrometry (SIFT-MS) etc.

Due to the compatibility of the apparatus 100 with analysing instruments and compounds, the apparatus 100 can be used for sampling for routine qualitative and quantitative volatile compound analysis. The apparatus 100, particularly the volume of the sample collection volume 114, can be selected for sampling of whole and end-tidal human and animal breath for the detection of disease markers from biological samples like exhaled breath. The apparatus 100 may similarly be used for outdoor and indoor air sampling for air quality monitoring by, for example, allowing the apparatus 100 to remain in a room with both closures 104, 106 open to permit the contents of the chamber 102 to mix with the air being sampled for a period sufficient for complete mixing. The apparatus 100, due to the silanised surface being capable of storing volatile compounds without reacting, for extended periods, may be suitable for use in ecological studies like chemical ecology, and so forth.

One or both closures 104, 106 may also incorporate a time or pressure/partial pressure sensor for automatically closing the valves or closures 104, 106. Thus, as exhalation force decreases sufficiently, the outlet closure 106 will close automatically and then the inlet closure 104 can be manually or automatically closed. The valve or actuating mechanism therefor can be a sliding or gate valve, knob actuated valve, button actuated valve or any other suitable type.

The apparatus 100 may further comprise side handles (not one of which is referenced 119 in FIG. 1)—e.g. on the chamber 102—to facilitate gripping by a subject during exhalation into the chamber 102. The apparatus 100 may further comprise integrated gas and/or volatile organic compound sensors 120 along with, or in, the chamber 102. These sensors can be used for real time identification of gases, biomarkers or components present in the samples. An LED indicator 122 may also be used to indicate the presence of a sample in the chamber 102.

The apparatus 100 is compact and portable. The apparatus 100 can therefore be used for collecting breath samples from the patient's/subject's place such as home, hospital ward, outpatient clinics or any other clinical setting. This apparatus 100 is reusable, thus reducing the cost of diagnosis significantly. For example, once used apparatus 100 can be washed with of 70% ethanol and double distilled water followed by autoclaving and drying in hot air oven. This cleansing procedure makes the apparatus 100 ready for the next breath collection.

The results obtained using the present apparatus 100 are highly reproducible and repeatable.

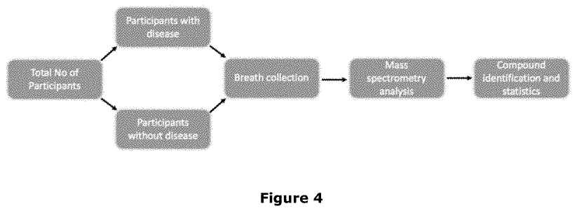

The apparatus 100 may be used for the identification of breath markers for specific ill-health conditions in patients. The assessment of the effectiveness of breath collection tube or apparatus 100 in separating the end tidal breath from the total tidal volume is critical. To study this, “breath profiles” of the ambient air and breath from subjects collected at the same place as the ambient air were sampled. FIG. 4 outlines the experimental procedure used for the study. The apparatus 100 proved suitable for end tidal breath collection while maintaining the ability to analyse samples without reacting with the internal glass surface of the sample collection chamber 102.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps. Moreover, features from different embodiments may be combined with other embodiments, features may be substituted for one another or removed, without departing from the present teachings.

The reference to any prior art in this specification is not, and should not be taken as, an acknowledgment or any form of suggestion that the prior art forms part of the common general knowledge.

1. An apparatus for collecting part of an exhaled breath, comprising:
a chamber comprising:
an internal surface defining a sample collection volume, that has been functionalised so that it does not interact chemically or physically with volatile components in the breath;
an inlet end through which the breath enters the sample collection volume; and
an outlet end through which the breath exits the sample collection volume;
two closures, one at each of the inlet end and the outlet end to control passage of the breath through the respective inlet end or outlet end, the closures being actuated to capture the part of the exhaled breath; and
a sampling port accessible from outside the chamber and in communication with the sample collection volume,
wherein the chamber has a longitudinal extent, and has a taper in its diameter toward each of the inlet end and outlet end.

2. An apparatus according to claim 1, being used for the collection of end tidal breath.

3. An apparatus according to claim 1, wherein the internal surface has been functionalised by being silanized.

4. An apparatus according to claim 3, wherein the chamber is formed from glass or any chemically inert materials.

5. An apparatus according to claim 1, wherein the sample collection volume is a predetermined internal volume that is less than a full exhalation volume.

6. An apparatus according to claim 5, wherein the predetermined volume is between about 30 mL and about 400 mL.

7. An apparatus according to claim 6, wherein the predetermined volume is between about 70 mL and about 350 mL.

8. An apparatus according to claim 7, wherein the predetermined volume is about 70 mL.

9. An apparatus according to claim 1, wherein the closure at the inlet end comprises a valve.

10. An apparatus according to claim 9, wherein the valve is a one-way valve to permit passage of breath into the chamber and to prevent passage of breath out of the chamber through the inlet end.

11. An apparatus according to claim 1, wherein the closure at the outlet end comprises a valve.

12. An apparatus according to claim 1, wherein at least one of the two closures comprises a vacuum stopper.

13. An apparatus according to is claim 1, wherein each of the two closures comprises a vacuum stopper.

14. An apparatus according to claim 1, wherein the closure at the outlet end operates independently of the closure at the inlet end.

15. An apparatus according to claim 14, wherein the sampling port is formed in one of the two closures.

16. An apparatus according to claim 14, wherein the sampling port is disposed between the inlet end and the outlet end.

17. An apparatus according to claim 14, wherein the sampling port comprises a gastight septum.

18. An apparatus according to claim 1, wherein the taper is selected to maintain laminar flow of breath through the chamber.

19. An apparatus according to claim 1, wherein the chamber is substantially cylindrical, having a longitudinal axis extending through both the inlet end and outlet end.
